(19) United States
(12) Patent Application Publication
(10) Pub. No.: US 2004/0087864 A1
(43) Pub. Date: May 6, 2004

(54) Method and apparatus for assessment and treatment of cardiac risk

(76) Inventor: Lawrence Grouse, Gig Harbor, WA (US)

Correspondence Address: ANTHONY H. HANDAL, KIRKPATRICK & LOCKHART, LLP, 599 LEXINGTON AVENUE, 31ST FLOOR, NEW YORK, NY, 10022-6030, US

(21) Appl. No.: 10/283,985

(22) Filed: Oct. 30, 2002

(51) Int. Cl. A61B 5/02

(52) U.S. Cl. 600/508

(57) ABSTRACT

A method for risk evaluation, determining treatment options, and the assessment of tested treatment options is implemented by the use of specialized computing device. Reducing likelihood that the inventive method will not be implemented on account of the difficulty of introducing a computer into a typical physician-patient clinical setting is mediated by implementation on a handheld device which can unobtrusively be used by the physician in the presence of the patient and with ease, and without being so obvious to disturb the patient into believing that judgments are being made by a computer. Treatment options are provided in suggestion form in order to avoid automatic implementation of suggestions without the necessary input of physician judgment. The above advantages are achieved without the inherent unreliability of personal computing systems by use of a dedicated computing device. Costs are controlled by a minimally sized display and input keypad.

### STATEMENT REGARDING FEDERALLY SPONSORED RESEARCH OR DEVELOPMENT

Not Applicable.

### BACKGROUND OF THE INVENTION

Because of its importance to a large segment of this population, or compared to other conditions, coronary heart disease has been the subject of particular attention during the 20th century from the standpoint of understanding the root causes of various diseases and what steps can be taken to prevent the same. A century ago, physicians generally did perceive a link between obesity and heart disease. However, the general view was that the large bulk of an obese individual represented an excessive load for an overburdened heart, in much the same way as excessive exercise during a short period of time could damage the heart.

Even by the middle of the 20th century, the protocol for lowering the incidence of coronary heart disease was relatively simple. Patients were told to eliminate, or at least reduce, smoking, and limit the intake of foods. As far as cardiac patients were concerned, calorie counting was the primary tool on the dietary front.

Gradually, however, the picture became increasingly complex. It emerged that cholesterol was an important factor in assessing the risk of coronary heart disease. Exercise, likewise, came to be recognized as an important factor in lowering cholesterol. Fats were also recognized as a dietary component which could be problematic. Accordingly, patients were counseled to reduce fat intake. It then emerged that all fats are not equal in terms of the potential damage that they can do to the heart. Differing impacts on the heart for unsaturated, saturated, monounsaturated and polyunsaturated fats came to be known. It was also learned that cholesterol, per se, is not the best indicator of risk, and that cholesterol in the body can be divided between HDL (high density lipoprotein) cholesterol, and LDL (low density lipoprotein) cholesterol. In addition, triglycerides were found to be a part of the factors involved in an assessment of risk of cardiac disease. Thus, the assessment of risk of cardiac heart disease reached a higher degree of complexity, when it was recognized that not all forms of cholesterol increase the risk of heart disease. In particular, it was recognized that, to the contrary, HDL cholesterol (a high density material packaged by the body for elimination and less likely to adhere to interior coronary sidewalls), if present in sufficient quantities, appeared to lower the risk of myocardial infarction and coronary death.

Still another complicating factor has been the availability of cholesterol-lowering medications. Many specialists in the field believe that such medications should be almost universally administered. Others question efficacy in such a diverse population, and point to cost concerns, which are no longer strangers to medical care.

Given the wealth of information data, the problem then became one of properly assessing the data. The Report of the National Cholesterol Educational Program sought to outline a strategy for primary prevention of coronary heart disease in persons with high levels of low-density lipoprotein (LDL) cholesterol (greater than or equal to 160 milligrams per deciliter) or those with borderline-high LDL cholesterol (130-159 milligrams deciliter) and multiple (i.e. two or more) risk factors.

The Second Report of the Expert Panel on Detection, Evaluation and Treatment of High Blood Cholesterol in Adults repeated this approach and added the intensive management of LDL cholesterol in persons with established chronic heart disease, setting a new lower LDL cholesterol goal of less than or equal to 100 milligrams per deciliter for chronic heart disease patients.

The Third Report of the National Cholesterol Education Program (NCEP) Expert Panel on Detection, Evaluation, and Treatment of High Blood Cholesterol in Adults (ATP III) attempted to deal with continuing difficulties being experienced by the medical community in assessing and evaluating all the new information and treatment options.

Thus, unlike other conditions, in the case of a great part of the population, the causes of heart disease are better understood today and effective prevention strategies are, at least from a theoretical viewpoint, available. Nevertheless, heart disease remains one of the leading health problems in the United States. The situation is exacerbated due to the fact that the prosperity, which our country now enjoys, increases the incidence of heart disease in the general population on account of the impact of richer diets and sedentary life styles.

ATP III builds upon the Second Report, and adds the facet of focusing on primary prevention in persons with multiple risk factors. In particular, for many persons with multiple risk factors, ATP III recommends aggressive LDL-lowering treatments as compared to the Second Report. In addition, numerous other refinements have been introduced, including treating persons with diabetes but without coronary heart disease as having a risk level comparable to that of persons with coronary heart disease. Likewise, the Framingham projections of ten-year absolute cardiac heart disease risk to identify patients with multiple risk factors for more intensive treatment, as well as to identify persons with multiple metabolic risk factors as candidates for intensified therapeutic lifestyle changes are implemented.

In accordance with ATP III, a complete glycoprotein profile including measurements of total cholesterol, LDL-cholesterol, HDL cholesterol and triglycerides is the preferred initial test. This compares with the prior approach of screening for total cholesterol and HDL cholesterol during the initial testing of a patient.

ATP III represents the latest attempt to logically approach the very complex interrelationships between relevant cardiac heart disease risk factors and balance the same in crafting an effective treatment regimen. The same is achieved using the Framingham factors. Alternative approaches implement a mathematical formula treating various risk factors or an estimation approach based on points.

More particularly, in accordance with the Adult Treatment Panel III (ATP III) guidelines for clinical cholesterol management, medical professionals are provided with a specific methodology to assess risk and improve coronary heart disease outcomes in their patients. A complex nine step approach has been detailed as part of a National Cholesterol Education Program spearheaded by the United States Department of Health and Human Services.

The first step is the determination of glycoprotein levels using a complete glycoprotein profile after a 9 to 12 hour patient fast. LDL-cholesterol is measured as the primary target of the therapy, with optimal levels being below 100 milligrams per deciliter. Total cholesterol levels are desirably below 200 milligrams per deciliter with HDL's desirably greater than 60 milligrams deciliter.

The next step is the identification of any clinical atherosclerotic disease, which carries a high risk of coronary heart disease.

The physician then determines the presence of other major risk factors (other than LDL-cholesterol). These include cigarette smoking, hypertension, and a family history of premature coronary heart disease (premature being defined as before the age of 55 in the case of a male first-degree relative and below the age of 65 in a female first-degree relative).

If there are two or more risk factors present, the Framingham tables are used to assess the ten-year (short-term) coronary heart disease risk, unless coronary heart disease or coronary heart disease risk equivalent (an event) is present.

In a fifth step, risk category is determined by establishing a goal for LDL-cholesterol level, determining the need for therapeutic lifestyle changes and determining whether a particular level of cholesterol-lowering drug therapy should be considered. More particularly, the establishment of LDL goals in ATP III are listed at less than 100 milligrams for deciliter for coronary heart disease risk equivalents or coronary heart disease risk greater than 20 percent. Where risk is below 20 percent and two or more risk factors are present, LDL goals are set at less than 130 milligrams deciliter. Finally, where there are one or fewer risk factors, listed LDL goals are below 160 milligrams deciliter.

The next step is to initiate therapeutic lifestyle changes if LDL cholesterol is above the goal. These include reduction in the dietary intake of saturated fats, introduction of viscous fiber into the diet and treatment with plant stanols/sterols, together with weight management and increased physical activity.

Step 7 involves consideration of drug therapy if LDL cholesterol exceeds the above step 5 levels. More particularly, and drug therapy should be considered together with implementation of therapeutic lifestyle changes for coronary heart disease and coronary heart disease problems. Alternatively, consideration should be given to ending drug therapy after three months of therapeutic lifestyle changes and an evaluation of progress.

The next step involves the identification of metabolic syndrome and treatment of the same, if present, after three months of therapeutic lifestyle changes. Determination of metabolic syndrome involves assessing abdominal obesity, high triglyceride levels, low HDL cholesterol levels, high blood pressure and testing for glucose. Different cutoff points for these factors are involved for men and women.

The last step is a treatment of elevated serum triglycerides with all ranges being below 150 milligrams deciliter and very high ranges extending above 500 milligrams deciliter. This is combined with treatment of elevated triglycerides and treatment of low HDL cholesterol.

As noted above, the evaluation of the risk of coronary heart disease is done using point scores associated with the age of the patient, total cholesterol, whether the patient is a smoker or non-smoker, the level of HDL cholesterol, and systolic blood pressure. In accordance with the Framingham points scored technique, points are edited and depending upon point total risk is estimated. For example, for men, 0 points carries a less than 1 percent risk of heart disease over a ten-year period, and 10 points carries a 6 percent risk of heart disease and 17 or more points carries a risk of heart disease greater than or equal to 30 percent. Assignation of point scores and assessment of risk on the basis of total points is different for women.

Alternatively, a mathematical formula developed on the basis of the Framingham Study may be used to assess risk.

As can be seen from the above, the determination of risk, determination of treatment strategy, testing treatment options and determination of alternate approaches, where necessary, has become a complex task. Moreover, the mathematics associated with the assessment of long-term risk, and the mental distraction and/or overload associated therewith is impeding the quality generation of patient-specific methodologies of treating the patient's condition. In addition, the increasing pressure to provide treatment to more and more patients, and the time pressures associated therewith is increasing the likelihood of less than optimal judgments by medical practitioners.

Particularly in the last few years, in addition to the difficulties posed by the complexity of the tasks required to provide quality medical care in the cardiac field, the dominating presence of managed care has exerted substantial economic pressure on the practice of medicine. Hospitals and doctors are being forced to regulate the amount of time being spent by an individual physician with his patient. The combination of time pressure, sometimes difficult patients, distractions, and other factors all contribute, together with the increased complexity of the problem, to the difficulty of a quality assessment of a patient's coronary heart disease risk, and development of an appropriate treatment strategy.

### SUMMARY OF THE INVENTION

Perhaps more seriously for the future, is the likelihood that the situation is expected to become increasingly complex in the future. For example, new research is beginning to indicate that certain LDL cholesterols are associated with extremely high levels of risk. Thus, in the future, additional tests will indicate the levels of these very high risk LDL cholesterols, and appropriate more complex weighting, or other risk evaluation methodology, will have to be applied, before treatment strategies, strategy testing and strategy amendments will have to be determined. Thus, the future appears to hold increasingly higher likelihood of coronary risk assessment complexity, economic and time pressures, and consequential higher likelihood of less than optimal treatment option determination and evaluation.

In accordance with the invention, risk evaluation, treatment options, and assessment of tested treatment options is implemented by the use of specialized computing device. In addition, reducing likelihood that the inventive method will not be implemented on account of the difficulty of introducing a computer into a typical physician-patient clinical setting is mediated by implementation on a handheld device which can unobtrusively be used by the physician in the presence of the patient and with ease, and without being so obvious to disturb the patient into believing that judgments are being made by a computer.

At the same time, in accordance with a preferred embodiment, treatment options are provided in suggestion form in order to avoid automatic implementation of suggestions without the necessary input of physician judgment.

It is an object of the present invention to achieve the above advantages without the inherent unreliability of personal computing systems. The same is achieved by a dedicated computing device. Costs are controlled by a minimally sized display and input keypad. The result is a computing device which also has very low power consumption, and thus extended battery-powered life, thus providing additional degrees of convenience and reliability.

In accordance with a particularly preferred embodiment of the invention, information on various patients is stored in random access memory contained in the inventive device and downloaded to a conventional personal computer by way of an infrared port, Blue Tooth communications protocol, a USB connection or other hard wired or wireless communications technique.

In accordance with the invention, a method of determining a regimen for the treatment of individuals with elevated risks of developing coronary heart disease comprises receiving and electronically storing risk factor information respecting the sex, age, blood chemistry and lifestyle of an individual, electronically executing an algorithm on said risk factor information, and displaying at least one result of said execution of said algorithm on said risk information.

An inventive method of reducing the risk of coronary heart disease in the general population comprises presenting on a handheld computing device a series of questions relating to risk factors for coronary heart disease respecting the sex, age, blood chemistry and lifestyle of an individual. The handheld computing device is used to receive and electronically store risk factor information input by a physician. An algorithm is electronically executed on a dedicated electronic device by using the input risk factor information. At least one result of said execution of the algorithm evaluating said risk information is output by the handheld device in the form of a message identifying a potential therapy for consideration.

The inventive method of reducing the risk of coronary heart disease in the general population, also contemplates the possible funding of distribution of dedicated electronic devices by selling advertising physically associated with the dedicated electronic devices. Such advertising may be associated with possible treatments for reducing the risk of coronary heart disease.

### BRIEF DESCRIPTION OF THE DRAWINGS

The inventive method and apparatus may be understood from the description below, taken together with the drawings, in which:

FIG. 1 illustrates an input device constructed in accordance with the present invention and which is adapted to implement the method of the present invention;

FIG. 2 is a view of the reverse side of the inventive device illustrated in FIG. 1;

FIG. 3 is an alternative embodiment of the inventive device;

FIGS. 4-18 illustrates the use of the inventive device to practice the inventive method during the data input phase;

FIG. 19 is a flow diagram illustrating the method of the present invention;

FIGS. 20-22 illustrate the inventive device during part of the data output phase of the inventive method; and

FIGS. 23-25 illustrate the alternative inventive device illustrated in FIG. 3 during data entry steps of the inventive method.

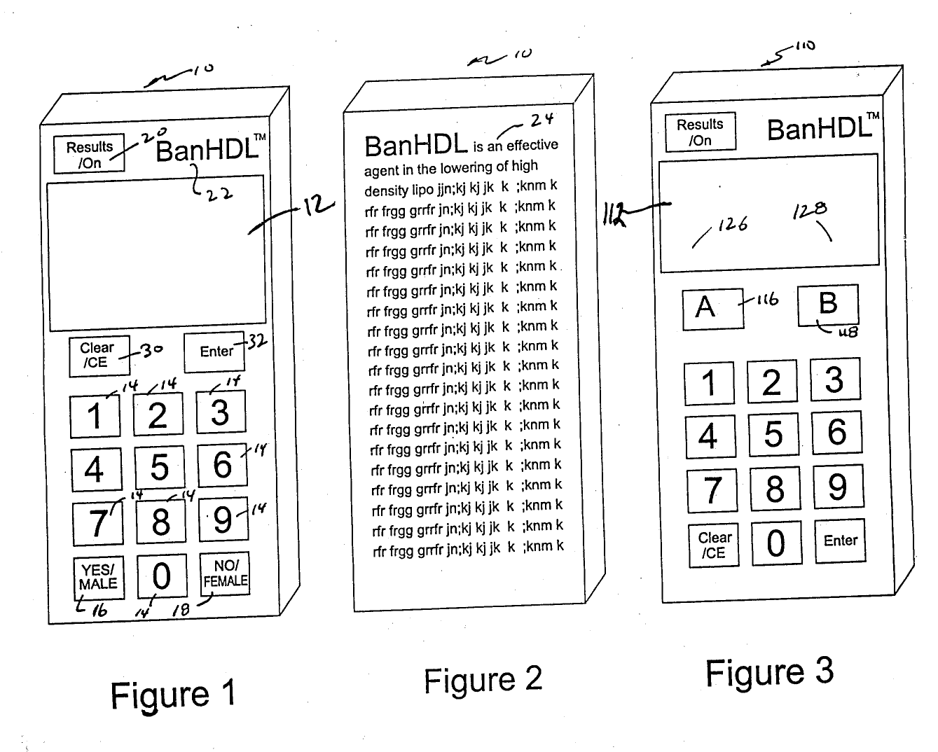

### DETAILED DESCRIPTION OF THE PREFERRED EMBODIMENTS

In accordance with the present invention, an inventive input device 10 is used to input data during implementation of the inventive method. Input device 10 includes a display 12 for indicating what information should be input into device 10, and for outputting results. The key pad is formed by a plurality of keys, including numerical keys 14, special-purpose input keys 16 and 18, and a “results” key 20.

In addition to the electronic functions implemented by keys 14-18, in accordance with the preferred embodiment of the invention, funding for the device is achieved through the use of advertising, in this case the inclusion of the trademark 22 of a drug “BanHDL”. In the instant case, the manufacturer of the drug BanHDL pays money or other consideration to the person or organization distributing the inventive input device 10. Alternatively, the inventive input device 10 may be distributed by the drug manufacturer. In addition to the above, and in order to build value into the inventive input device 10, information respecting the drug, or other products maybe provided in the form of alphanumeric printing 24 on the reverse side of data input device 10, as illustrated in FIG. 2. Such alphanumeric printing 24 may be imprinted in any fashion, such as a silk screen technique, by being adhered after being printed on a self-adhesive transparent or opaque label using a computer printer, or the like, or other process.

In accordance with the invention, the inventive data input device is also provided with a “clear/clear entry” key 30 which when depressed once clears an entry, and when pressed twice clears all entries and returns the user to the initial screen. And “enter” key 32 is depressed in order to enter information input into the system using keys 14.

In accordance with the present invention, alternative arrangements of keys and screen and the like, may be implemented, as illustrated, for example, in FIG. 3, by data input device 110, which will be described in greater detail below.

Generally, however, in this embodiment, the output of screen 112 is set up to cooperate with keys 116 and 118 which may or may not be marked with alphanumeric data. Special purpose keys 16 and 18 have been replaced by keys 116 and 118. More particularly, in accordance with the invention it is contemplated that region 126 will carry information respecting the function of special-purpose key 116, and region 128 will carry information respecting the function of special-purpose key 118.

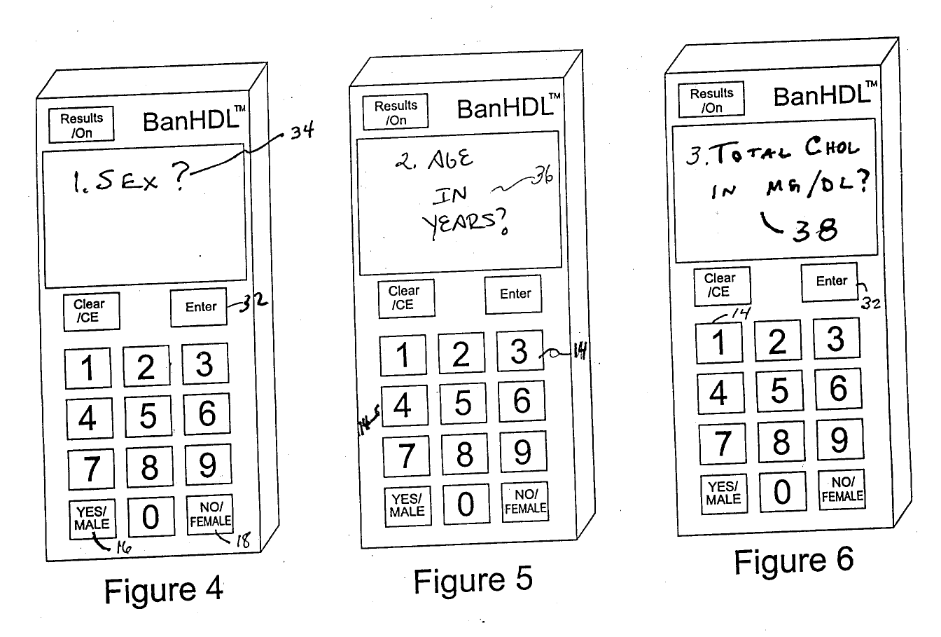

Referring to FIGS. 4-18, the inventive method commences with the actuation of key 20. In accordance with the preferred embodiment, keys 14-20 are pushbutton keys and may be activated by simply being pressed. When key 20 is pressed, the first data input screen is presented, as illustrated in FIG. 4.

Alternatively, when key 20 is pressed, optionally, a welcome screen may be presented saying, for example: “Welcome to risk calculation” or, perhaps more elaborately: “Welcome. We will guide you step-by-step to calculate risk of cardiac heart disease.” After a period of time, the welcome screen will disappear and the first data input screen will be presented.

In accordance with the invention, the inventive data input device 10 facilitates the assessment by a physician of a patient's coronary heart disease risk factors, and simply calculates 10-year risk coronary heart disease based on the individual patient's coronary heart disease risk factors, as well as to establish a risk category and suggest an LDL-cholesterol goal for the patient, as well as help the physician determine the best treatment options to reach that goal and thus reduce the risk of coronary heart disease events.

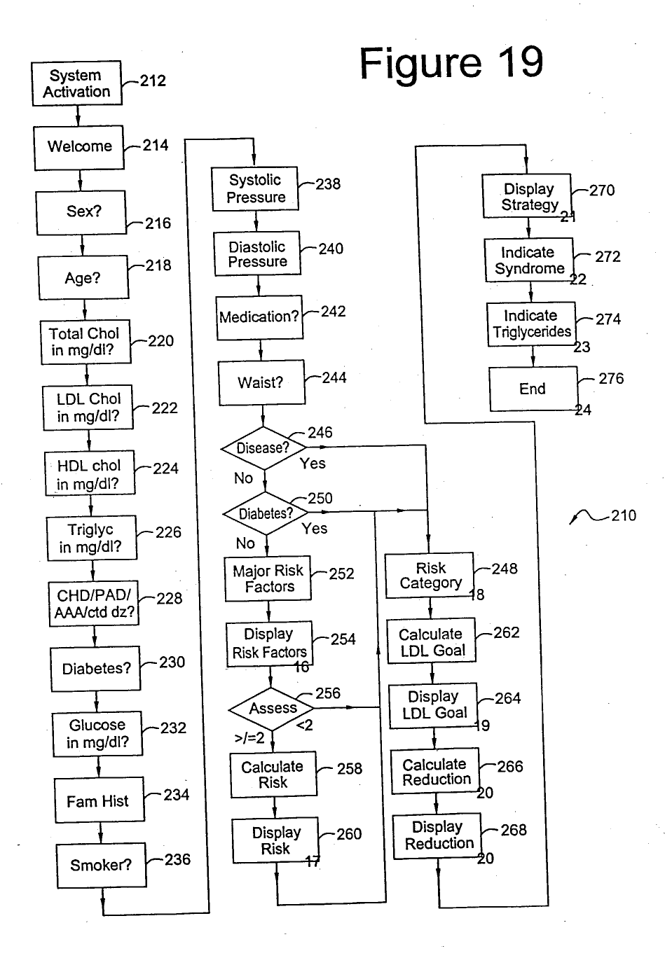

As illustrated by the data input screens illustrated in FIGS. 4-18, the inventive process 210 (FIG. 19) begins by collecting data from the patient, initially by way of physical examination, blood tests and the like. These include such things as waste measurements, weight, LDL-cholesterol, HDL cholesterol, total cholesterol, triglycerides, whether the patient smokes, and so forth, as recommended by the ATP III guidelines and the general practice of the physician, which items of data are utilized by the method and apparatus of the present invention as detailed below.

The inventive calculator, once activated by depression of key 20, at step 212, and the optional presentation of a welcome screen at step 214, begins by asking the physician a series of questions about the patient's laboratory results, lifestyle risk factors, medical history, and so forth, as per the above collection of information, as will be detailed below. The requested information is input into the data input device 10 using the special purpose and numeral keys as appropriate. If the data has been successfully entered, the physician then presses the “Enter” key 32 to enter the information and cause data entry device 10 to display the next screen. The “Clear” key may be used to clear the current screen, if necessary, by being pressed once. Pressing the “Clear” key more than once causes the system to be completely cleared and to present the welcome screen to the physician for the input of a complete set of data as detailed below.

Referring to the data input screens illustrated in FIGS. 4-18, at the first data input screen display 34 (FIG. 4), presented at step 216, the system asks the physician to input the sex of the patient. The system may simply display: “1. Sex?”. A more elaborate message may be used on the screen (and any of the other screens that are described below), such as: “Enter patient's sex and press ‘Enter’”. In response, the physician depresses key 16, if the patient is male, or key 18, if patient is female. Enter key 32 is then pressed to enter the data into the random access memory of data input device 10, and the next screen is presented at the next step.

In accordance with the present invention, each of the screens is assigned a screen number (in the case of the screen of FIG. 4, the screen number “1”). In the case of FIGS. 4-18, the screen number appears in the form of a question number (in the case of FIG. 4, the question number is “1”), which appears on the screen together with the request for information (i.e. the “question”). In similar fashion, when data is output by the system, such data is also associated with a screen number which is displayed on the display. The function of the screen number is to identify the screen by number allowing correlation of printed instructional information to each particular screen, as appears more fully below.

Referring to FIG. 5, at step 218, the system presents the next data input screen display 36, reading: “2. Age in Years?” (question number 2 at screen number 2). In response, the physician enters in the age of the patient using numerical keys 14. Enter key 32 is then depressed to enter the data into the random access memory of data input device 10, and the next screen is presented at the next step.

Referring to FIG. 6, at step 220, the system presents the next data input screen display 38, reading: “3. Total Chol in mg/dl?” (question number 3 at screen number 3). In response, the physician enters the total cholesterol level of the patient in mg per dl using keys 14. Enter key 32 is then depressed to enter the data into the random access memory of data input device 10, and the next screen is presented at the next step.

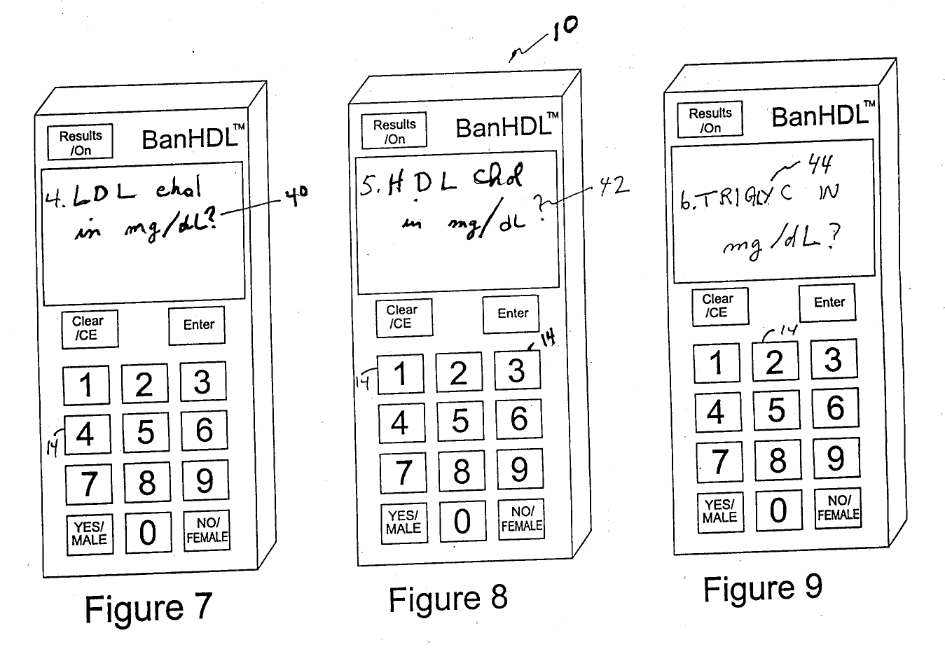

Referring to FIG. 7, at step 222, the system presents the next data input screen display 40, reading: “4. LDL Chol in mg/dl” (question number 4 at screen number 4). In response, the physician enters the cholesterol of the patient in mg per dl using keys 14. Enter key 32 is then depressed to enter the data into the random access memory of data input device 10, for later use by the system together with the other data input by the physician, as will be described in detailed below. After this, the next screen is presented at the next step.

Referring to FIG. 8, at step 224, the system presents the next data input screen display 42, reading: “5. HDL chol in mg per dl?” (question number 5 at screen number 5). In response, the physician enters the HDL cholesterol level for the patient using keys 14. Enter key 32 is then depressed to enter the data into the random access memory of data input device 10, and the next screen is presented at the next step.

Referring to FIG. 9, at step 226, the system presents the next data input screen display 44, reading: “6. Triglyc in Mg/Dl?” (question number 6 at screen number 6). In response, the physician enters the triglycerides level of the patient using keys 14. Enter key 32 is then depressed to enter the data into the random access memory of data input device 10, and the next screen is presented at the next step.

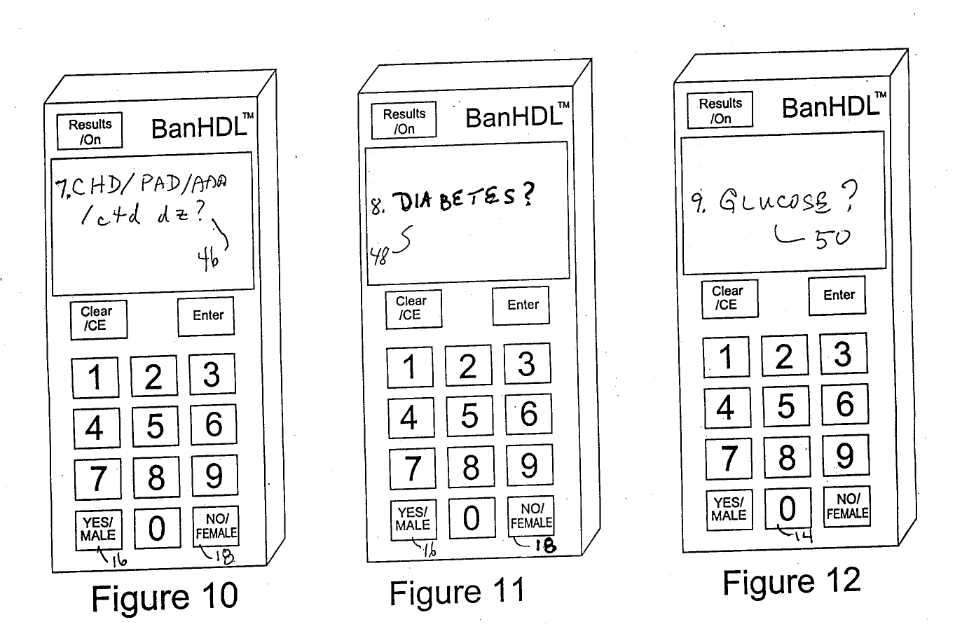

Referring to FIG. 10, at step 228, the system presents the next data input screen display 46 reading: “7. CHD/PAD/AAA/ctd dz?” (question number 7 at screen number 7). The object of this query is to determine whether the patient has been diagnosed with any one of the following conditions: coronary heart disease (CHD), peripheral arterial disease (PAD), abdominal aortic aneurysm (AAA) or carotid artery disease (symptomatic, or asymptomatic with greater than 50 percent stenosis) (ctd dz). In response, the physician enters a “Yes” or “No” for the patient using keys 16 and 18, depending upon whether or not any one of these conditions has been diagnosed in the patient. Enter key 32 is then depressed to enter the data into the random access memory of data input device 10, and the next screen is presented at the next step.

Referring to FIG. 11, at step 230, the system presents the next data input screen display 48, reading: “8. Diabetes?” (question number 8 at screen number 8). In response, the physician enters whether the patient has diabetes using either the “Yes” and “No” keys 16 and 18. Enter key 32 is then depressed to enter the data into the random access memory of data input device 10, and the next screen is presented at the next step. In accordance with an alternative embodiment of the invention, whenever “Yes” or “No” keys 16 and 18 are depressed, the next screen may be presented without the necessity of pressing enter key 32.

Referring to FIG. 12, at step 232, the system presents the next data input screen display 50, reading: “9. Glucose?” (question number 9 at screen number 9). In response, the physician enters the fasting glucose level of the patient using keys 14. Enter key 32 is then depressed to enter the data into the random access memory of data input device 10, and the next screen is presented at the next step.

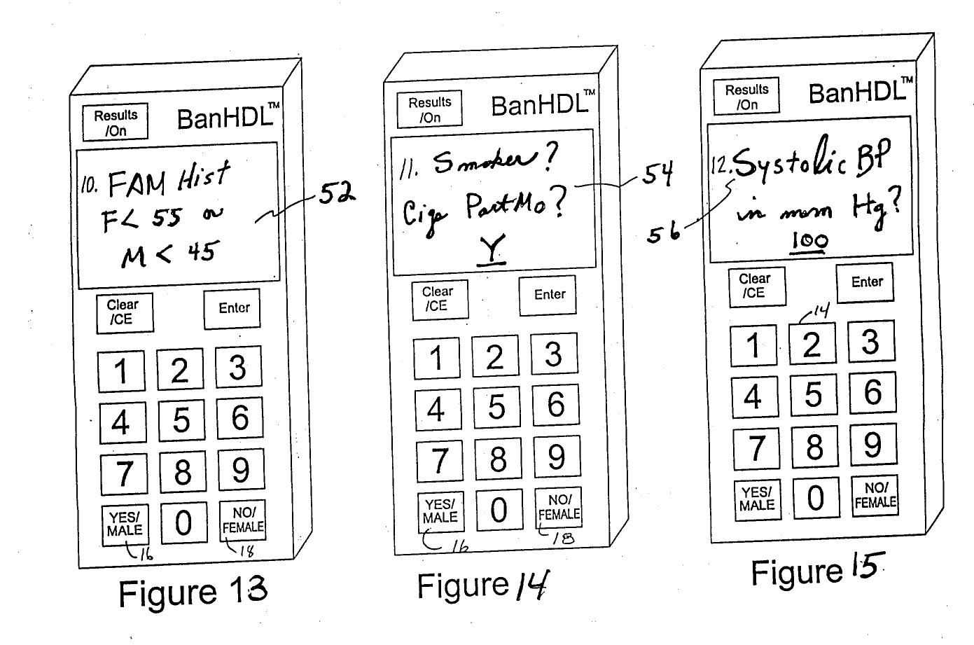

Referring to FIG. 13, at step 234, the system presents the next data input screen display 52, reading: “10. Fam Hist F<55 or M<45” (question number 10 at screen number 10). In response, the physician enters a “Yes” by using keys 16 if a male first-degree relative (father or brother) of the patient had a heart attack before the age of 45, or a female first-degree relative (mother or sister) of the patient had a heart attack before the age of 55 using key 16, as per the ATP III guidelines. Otherwise, a “No” is entered into the system using key 18. Enter key 32 is then depressed to enter the data into the random access memory of data input device 10, and the next screen is presented at the next step.

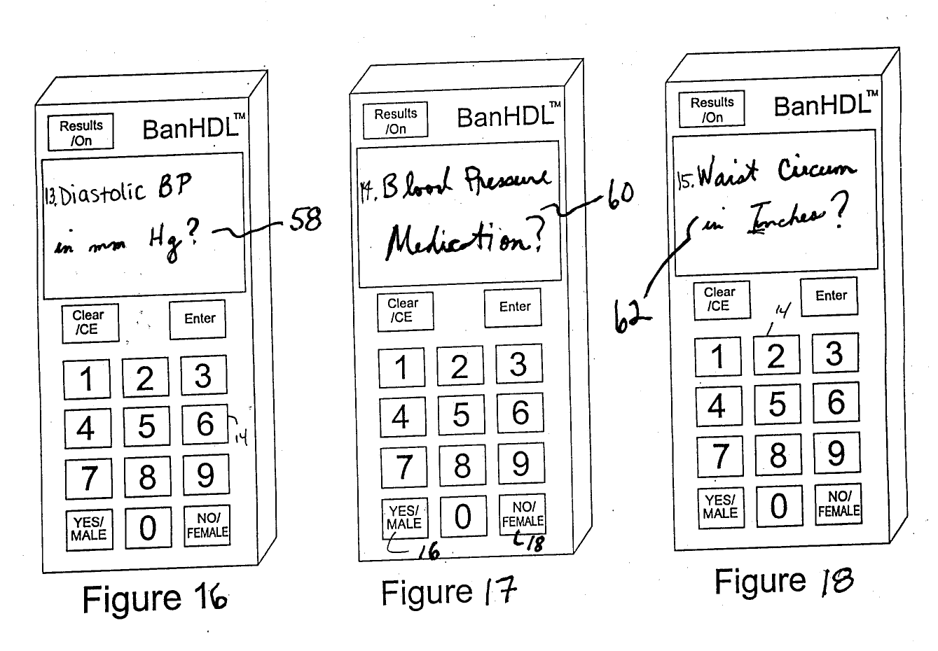

In accordance with a preferred embodiment of the invention, whenever the “yes” or “no” buttons are depressed or a number entered, the same appears on screen 12, as illustrated in FIGS. 14 and 15, respectively.

Referring to FIG. 14, at step 236, the system presents the next data input screen display 54, reading: “11. Smoker? Cigs Past Mo?” (question number 11 at screen number 11). In response, the physician enters a “Yes” or “No” for the patient, depending upon whether the patient has smoked cigarettes within the last month. This is done using keys 16 and 18. Enter key 32 is then depressed to enter the data into the random access memory of data input device 10, and the next screen is presented at the next step.

Referring to FIG. 15, at step 238, the system presents the next data input screen display 56, reading: “12. Systolic BP in mm Hg?” (question number 12 at screen number 12). In response, the physician enters the systolic blood pressure of the patient in millimeters of Hg using keys 14. Enter key 32 is then depressed to enter the data into the random access memory of data input device 10, and the next screen is presented at the next step.

Referring to FIG. 16, at step 240, the system presents the next data input screen display 58, reading: “13. Diastolic BP in mm Hg?” (question number 13 at screen number 13). In response, the physician enters the diastolic blood pressure of the patient in millimeters of Hg using keys 14. Enter key 32 is then depressed to enter the data into the random access memory of data input device 10, and the next screen is presented at the next step.

Referring to FIG. 17, at step 242, the system presents the next data input screen display 60, reading: “14. Blood Pressure Medication?” (question number 14 at screen number 14). In response, the physician enters a “Yes” or “No” using keys 16 or 18, respectively, depending upon whether the patient is on medication to reduce high blood pressure or not. Enter key 32 is then depressed to enter the data into the random access memory of data input device 10, and the next screen is presented at the next step.

Referring to FIG. 18, at step 244, the system presents the next data input screen display 62, reading: “15. Waist Circum in Inches” (question number 15 at screen number 15). In response, the physician enters the circumference of the waist of the patient in inches using keys 14. Enter key 32 is then depressed to enter the data into the random access memory of data input device 10, and the next screen is presented at the next step.

When the enter key is pressed to enter the waist of the patient, and, optionally (depending upon design specifications) the results key 20 is pressed, the system proceeds to step 246. At step 246, the system, in accordance with the inventive method, retrieves information input in response to question 7 at screen number 7. If the answer which the physician entered into the system for question number 7 was positive, that is that the patient had been diagnosed with coronary heart disease (CHD), peripheral arterial disease (PAD), abdominal aortic aneurysm (AAA) or carotid artery disease (symptomatic, or asymptomatic with >50 percent stenosis) (ctd dz), the system proceeds to step 248. At step 248 risk is assessed in accordance with the method which will be detailed below.

If, after retrieving the answer to question 7 from random access memory, it is determined that such disease is not exist, the system proceeds to step 250, where the system consults random access memory to determine whether the patient has been diagnosed with diabetes. In the answer entered by the physician is that the patient has been diagnosed with diabetes, the system proceeds to step 248 for a determination of risk. If the answer is “No”, the system proceeds to step 252 to identify and assess major risk factors.

At step 252, the system determines the number of major risk factors by adding points. More particularly, if the responses to questions one and two show that the patient is a male over 45 or a female over 55, one point is assigned and added to the total of other applicable points. If the data entered into the system by the physician in response to question 5 indicates that the HDL cholesterol of the patient is below 40, another point is added to the total. However, if the HDL cholesterol is greater than or equal to 60, a point is subtracted from the total. In similar fashion, if the answer to question 10 shows a family history of early heart disease, another point is headed to the total. If the patient is a smoker, as indicated by the answer entered into the system in response to question 11, another point is added to the total. Finally, if the answer to question 12 indicates that systolic blood pressure is above 140, or that diastolic blood pressure is about 90, or that the patient is on blood pressure medication to lower blood pressure, another point is added to the total. Thus, if the patient is male and above 55, has an HDL cholesterol level below 40 milligrams per dl, has a family history of heart disease, is a smoker and has a systolic blood pressure above 140, the total is five major risk factors and the same is disclosed, at step 254, on the display screen 12 of input device 10, which displays “16. Five major risk factors” (screen number 16).

When results key 20 is pressed again, if the number of risk factors displayed on screen number 16 is less than two, the system proceeds to step 248. If the number risk factors displayed on screen number 16 is greater than two, the system proceeds to step 258.

At step 258 the ten-year risk of heart disease events (myocardial infarction and CHD death) is calculated using one of two equations, depending upon the sex of the patient. In particular, if the patient is a male, the probability of myocardial infarction and CHD death within the ten year period after which the data is collected is calculated using the equation:

exp(X−172.3002)

probability=1−0.9402,

where:

X=52.009610*1n(AGE)+20.014077*1n(TOTALCHOLESTEROL)−0.905964*1n(HDL CHOLESTEROL)+1.305784*1n(SYSTOLIC BP)+0.241549 (BP MEDS?)+12.096316 (SMOKING?)−4.605038*1n(AGE)*1n(TOTAL CHOLESTEROL)−2.843670*1n(AGE)*(SMOKING?) −2.933230*(1n(AGE)²)

The variable BPMEDS? is given the value 1 if the patient is on blood pressure lowering medicine, otherwise it is zero. The variable SMOKING? is given the value 1 if the patient smokes, otherwise it is 0.

It is noted that if the age of the male patient is greater than 70, then the term 2.843670*1n(AGE)*(SMOKING?) becomes 2.843670*1n(70)*(SMOKING?).

If the patient is female, then the probability of a coronary heart disease incident within the next ten years is calculated using the formula:

exp(X−146.5933)

Probability=1−0.98767,

X=31.764001*1n(AGE)+22.465206*1n(TOTAL CHOLESTEROL)−1.187731*1n(HDL CHOLESTEROL)+2.552905*1n(SYSTOLIC BP)+0.420251*1n(BP MEDS?)+13.075430(SMOKING?) −5.060998*1n(AGE)*1n(TOTAL CHOLESTEROL)−2.996945*1n(AGE)*(SMOKING?)

If the age of the female patient is greater than 78, then the term 2.996945*1n(AGE)*(SMOKING?) becomes 2.996945*1n(78)*(SMOKING?).

The symbol “*” is used herein to denote multiplication.

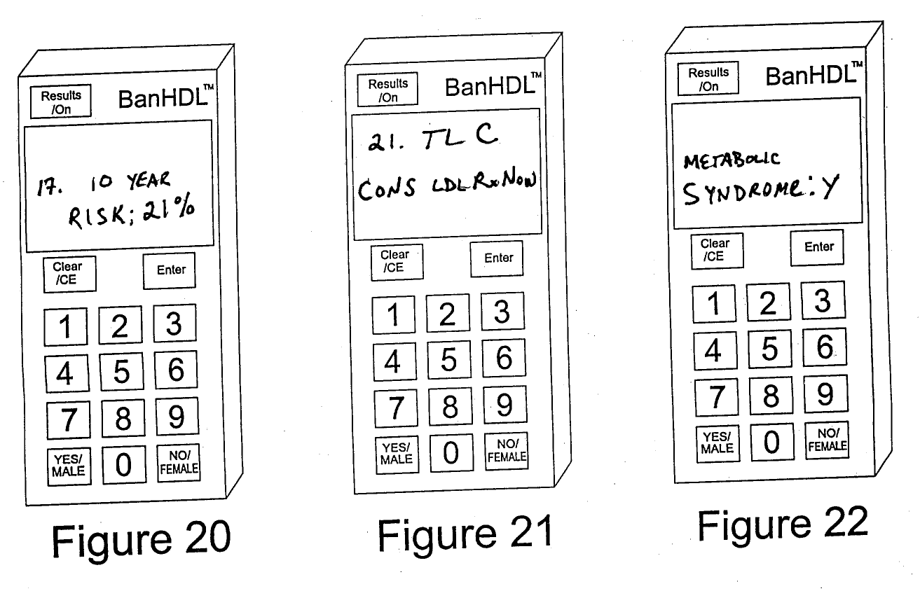

After the ten-year probability of heart disease events (myocardial infarction and CHD death) is calculated, the same is multiplied by 100 to show the percentage of risk and shown, at step 260, on display screen 12, as screen number 17, for example: “17.10 Year Risk: 21%”, as illustrated in FIG. 20. The system then proceeds to step 248 for the determination of risk category.

In accordance with the invention, one of three risk categories, as defined by ATP III, are displayed by the system after results key 20 is depressed. More particularly, risk categories are defined as category 1 denoting the highest risk, category 2 denoting serious risk, and category 3 denoting relatively low risk.

Risk is determined in accordance with ATP III guidelines based on a four rule algorithm:

The first rule is that if the answer to question 7 (chronic heart disease or risk equivalent) is “Yes” or the answer to question 8 (diabetes) is “Yes” then the risk category is 1.

The second rule is that if the answer to question 7 (chronic heart disease or risk equivalent) is “No” and the answer to question 8 (diabetes) is “No” and the sum from Screen 16 (the number of major risk factors) is less than 2 then the risk category is 3.

The third rule is that if the answer to question 7 (chronic heart disease or risk equivalent) is “No” and the answer to question 8 (diabetes) is “No” and the sum from Screen 16 (the number of major risk factors) is greater than or equal to 2 and the calculation from Screen 17 (10-year risk) is greater than 20 percent, then the risk category is 1.

The fourth rule is that if the answer to question 7 (chronic heart disease or risk equivalent) is “No” and the answer to question 8 (diabetes) is “No” and the sum from Screen 16 (the number of major risk factors) is greater than or equal to 2 and the calculation from Screen 17 (10-year risk) is greater than or equal to 20%, then the risk category is 2.

After risk category has been determined at step 248 and displayed at screen 18, for example in the case of a category 3 risk as “18. Risk Category 3”, the same is displayed on input device 10 at step 248. After results key 20 has been pressed again, the system then proceeds to calculate the LDL-cholesterol goal at step 262 and display the LDL-cholesterol goal in ml/deciliter at step 264. The LDL-cholesterol goal is determined in accordance with ATP III guidelines based on a three rule algorithm:

The first rule is that if the risk category is 1, then the LDL-cholesterol goal is less than 100 mg per deciliter.

The second rule is that if the risk category is 2, then the LDL-cholesterol goal is less than 130 mg per deciliter.

The third rule is that if the risk category is 3, the LDL-cholesterol goal is less than 160 mg per deciliter.

By way of example, if the patient has a risk category of 2, then display screen 12 presents screen number 19 as: “19. LDL goal <100 mg/dL”.

When device 10 is showing screen number 19, the LDL-cholesterol goal, pressing of the results key 20 causes the system, i.e. the electronics contained within device 10, to calculate the percentage of LDL-cholesterol reduction required to bring the patient from the current level of LDL-cholesterol to the goal. After the calculation is completed, screen number 20 is presented on display screen 12 at step 266. For example, if the patient must reduce LDL-cholesterol by 25 percent, screen number 20 reads: “20. 25% LDL reduction”. Depression of results key 20 during display of screen number 20 causes the system to execute a nine rule algorithm (in accordance with ATP III guidelines) which causes the system to determine ATP III recommended therapeutic options and present the same, at step 270, as screen number 21 on display screen 12:

The first rule is that if the risk category is 1 and the patient's LDL is less than or equal to 100 mg/dL, then screen number 21 reads “21. TLC, cons LDL Rx now.” Note use of the term “cons” (meaning “consider”)(in this case consider LDL lowering medication), as it is the objective of the invention to spark physician thought, and not provide a substitute therefore. Such a display is shown in FIG. 21.

The second rule is that if the risk category is 1 and the patient's LDL is greater than 100 mg/dL, then screen number 21 reads “21. TLC, LDL Rx opt”. “Opt” means optional; in the instant case, LDL lowering medication is optional.

The third rule is that if the risk category is 2 and the patient's LDL is greater than or equal to 160 mg/dL and the patient's 10-year risk<10%, then screen number 21 reads “21. TLC, cons LDL Rx 3 mo>160”.

The fourth rule is that if the risk category is 2 and the patient's LDL is greater than or equal to 130 mg/dL, and the patient's 10-year risk of a cardiac event, as defined above, 10-20%, then screen number 21 reads “21. TLC, cons LDL Rx 3 mo>129”, directing the physician to direct therapeutic life style changes and cholesterol lowering drugs in three months if the LDL does not drop below 130.

The fifth rule is that if the risk category is 2 and the patient's LDL is in the range 130-159 mg/dL and the patient's 10-year risk is less than 10% , then screen number 21 reads “21. TLC”.

The sixth rule is that if the risk category is 2 and the patient's LDL is greater than 130 mg/dL, then screen number 21 reads “21. LDL @ goal, followup”.

The seventh rule is that if the risk category is 3 and the patient's LDL is greater than or equal to 190 mg/dL, then screen number 21 reads “21. TLC cons Rx 3 mo>190 opt>159”, indicating more likely need of LDL medication if the LDL is 160 or higher and strong likelihood of the need from LDL cholesterol lowering drugs if the LDL is above 190. The eighth rule is that if the risk category is 3 and the patient's LDL is in the range 160-189 mg/dL, then screen number 21 reads “21. TLC, LDL Rx opt 3 mo>159”.

The ninth rule is that if the risk category is 3 and the patient's LDL is less than 160 mg/dL, then screen number 21 reads “21. LDL @ goal, followup”.

When the screen number 21 is being displayed, results key 20 may be depressed again, which causes the system to determine whether or not the patient is suffering from metabolic syndrome and display results in the form of screen number 21 at step 272 with a display screen which reads “22. Metabolic syndrome: Y” (as illustrated in FIG. 22) or “22. Metabolic syndrome: N”, respectively.

The presence of metabolic syndrome is determined if three or more of the following conditions apply to the patient on the basis of the data entered by the physician into device 10 and stored in the random access memory of device 10:

Condition 1: The answer to question 1 (sex) is male and the answer to question 5 (HDL) is greater than 40 or the answer to question 1 (sex) is female and the answer to question 5 (HDL) is greater than 50.

Condition 2: The answer to question 1 (sex) is male and the answer to question 15 (waist circumference) is greater than 40 inches or the answer to question 1 (sex) is female and the answer to question 15 (waist circumference) is greater than 35 inches.

Condition 3: The answer to question 6 (triglycerides) is greater than or equal to 150.

Condition 4: The answer to question 19 (fasting glucose) is greater than or equal to 110.

Condition 5: The answer to question 12 (systolic b.p.) is greater than or equal to 130 or the answer to question 13 (diastolic b.p.) is greater than or equal to 85.

If more than three of the above conditions are present, in accordance with ATP III guidelines, then the display on display screen 12 is: “22. Metabolic syndrome: Y”. If, on the other hand, less than three of the above conditions are present, then the display on display screen 12 reads: “22. Metabolic syndrome: N”.

When screen number 22 is being displayed on display screen 12, results key 20 may be depressed again, which causes the system to determine whether the level of triglycerides for the patient is high, borderline high or normal. This is done with three rules:

The first rule is that if the answer to question 6 (triglycerides) is greater than or equal to 200 mg/dL, then screen number 23 reads: “Triglyc: High”.

The second rule is that if the answer to question 6 (triglycerides) is in the range 150-199 mg/ dL, then screen number 23 reads: “Triglyc: Borderline High”.

The third rule is that if the answer to question 6 (triglycerides) is greater than 150 mg/dL, then screen number 23 reads: “Triglyc: Normal”.

When screen number 23 is being displayed on display screen 12, results key 20 may be depressed again, which causes the system to display: “End”. This indicates to the physician that the physician has completed the cycle of giving and receiving information. In the course of using the inventive device 10, it is contemplated in accordance with the invention that the physician will have on hand the patient's chart in order that the relevant information may be entered into device 10 during the data entry portion of the inventive process. Likewise, during the portion of the inventive process where the results key is being depressed and the physician is being provided with information, it is contemplated in accordance with the present invention that the physician will be entering onto the patient's chart his treatment decisions. Likewise, it is contemplated that the patient's chart may be an electronic document and the entry of the information into the chart will automatically generate a memorandum to the chart to the patient with and containing the recommended course of treatment.

In accordance with the invention, it is contemplated that the inventive input and output device 10 will be accompanied by an instructional card intended to guide the physician in the use of the inventive device 10. A suitable text for use on an instructional card, and which may further elaborate on the above, may read as follows:

With the touch of a few buttons, the ATP III calculator helps you assess an individual patient's coronary heart disease (CHD) risk factors, calculate the 10-year risk for CHD events (myocardial infarction and CHD death) in patients with two or more CHD risk factors, establish a risk category and LDL cholesterol goal for the patient, and determine the best treatment options to reach that goal and reduce the risk for CHD events.

The calculator summarizes and makes accessible the risk calculations and treatment recommendations contained in the Adult Treatment Panel III (ATP III) guidelines for clinical cholesterol management, which were developed by the National Heart, Lung, and Blood Institute's National Cholesterol Education Program. The calculation of CHD risk uses risk equations derived by the Framingham Heart Study.

Step 1: Enter Patient Information

The calculator begins by asking you a series of 15 questions about your patient's laboratory results, lifestyle risk factors, and medical history. Input the requested information using the “A,” “B,” and numeral keys as appropriate, and press the “Enter” key to enter the information and move to the next screen. Use the “Clear” key to clear the current screen if necessary.

Questions 3, 4, 5, and 6: Enter your patient's total cholesterol, LDL, HDL, and triglyceride levels, respectively.

Question 7: Enter “A” if your patient has been diagnosed with any of the following conditions: coronary heart disease (CHD), peripheral arterial disease (PAD), abdominal aortic aneurysm. (AAA), or carotid artery disease (symptomatic, or asymptomatic with >50% stenosis) (ctz dz).

Question 8: Enter “A” if your patient has been diagnosed with diabetes.

Question 9: Enter your patient's fasting glucose level.

1. Question 10: Enter “A” if your patient's father or brother developed CHD before age 55, or the patient's mother or sister before age 65.

Question 11: Enter “A” if your patient has smoked any cigarettes within the last month.

Question 14: Enter “A” if your patient is currently taking medication for high blood pressure.

Question 15: Enter your patient's waist circumference, in inches.

Step 2: Find Results

Press the “Results” key. You will be led through a sequence of screens depending on the answers you provided to the questions above. (Throughout this sequence, you can also use the “Review” key to return to the previous results screen.)

If your patient has established CHD or conditions that confer a risk of CHD events equal to that of established CHD (vascular disease, diabetes)—that is, if you answered “Yes” to Question 7 or 8—the calculator immediately takes you to Screen 18 and assigns the patient to Risk Category 1, indicating high risk for CHD (see below).

If you answered “No” to both Questions 7 and 8, the calculator determines how many major risk factors for CHD (other than LDL cholesterol level) your patient has [Screen 16].

If you are in Screen 16, press the “Results” key again.

If your patient has fewer than two risk factors, the calculator takes you directly to Screen 18 and assigns the patient to Risk Category 3, indicating low risk for CHD (see below).

If your patient has 2 or more risk factors, the calculator determines your patient's 10-year CHD risk [Screen 17]. (NOTE: The calculation of 10-year risk for CHD is intended to be performed before initiating cholesterol-lowering therapy. The calculator is not intended for assessing 10-year CHD risk in patients who are already on treatment or for tracking changes in CHD risk over time.)

If you are in Screen 17, press the “Results” key again to place your patient in one of three CHD risk categories (1=High; 2=Moderate, 3=Low) [Screen 18].

Once assigned to a Risk Category [Screen 18], all patients are taken through the same series of remaining screens:

Press the “Results” key again. Based on the patient's risk category, the calculator assigns an LDL goal for the patient [Screen 19].

Press the “Results” key to determine the percent reduction necessary to achieve the patient's LDL goal [Screen 20].

Press the “Results” key to determine the recommended treatment to achieve the patient's LDL goal [Screen 21]. The recommended treatment depends on the patient's risk category and current LDL level.

For all patients with elevated LDL cholesterol, the recommended treatment includes TLC, or therapeutic lifestyle changes. These consist of:

TLC diet

Saturated fat<7% of calories

Cholesterol<200 mg/day

Consider increased viscous (soluble) fiber (10-25 g/day) and plant

stanols/sterols (2 g/day) as therapeutic options to enhance LDL lowering

Weight management

Increased physical activity

For patients with LDL levels above certain cutpoints for their Risk Category, the treatment recommendations also encourage you to consider LDL-lowering drug treatment (“LDL Rx” on the screen). A summary of drug classes, available doses, and other information about LDL-lowering drug treatment is found in the ATP III Guidelines At-A-Glance Quick Desk Reference. The decision to use drug treatment is a matter for the physician's clinical judgement and discussion with the patient. The best choice of drug depends on the individual patient's medical history and percent LDL reduction necessary.

The recommendations concerning LDL-lowering drug treatment that appear on the calculator screen are as follows:

cons LDL Rx now—Consider LDL-lowering drug treatment now.

cons LDL Rx 3 mo>129—Consider LDL-lowering drug treatment if the patient's LDL is greater than 129 mg/dL after 3 months of TLC.

cons LDL Rx 3 mo>160—Consider LDL-lowering drug treatment if the patient's LDL is greater than 160 mg/dL after 3 months of TLC.

cons Rx 3 mo>190 opt>159—Consider LDL-lowering drug treatment if the patient's LDL is greater than 190 mg/dL after 3 months of TLC. LDL-lowering drug treatment is optional if the patient's LDL is greater than 159 mg/dL after 3 months of TLC.

LDL Rx opt—LDL-lowering drug treatment is optional.

LDL Rx opt 3 mo>159—LDL-lowering drug treatment is optional if the patient's LDL is greater than 159 mg/dL after 3 months of TLC.

LDL @ goal, followup—The patient's LDL is at goal for his or her Risk Category. Nevertheless, you should recommend that the patient undertake increased physical activity and other lifestyle changes as appropriate. Control of other risk factors and other follow-up as appropriate are also recommended.

Press the “Results” key to determine whether your patient has the characteristics of Metabolic Syndrome [Screen 22]. Treatment for this condition focuses first on weight control and increased physical activity. Further details are provided in the ATP III Guidelines At-A-Glance Quick Desk Reference, Step 8.

Press the “Results” key to determine whether your patient has high triglycerides [Screen 23]. For patients with high triglycerides>/=200 mg/dL, after the LDL goal is met therapy to reduce non-HDL cholesterol should be considered. Further treatment details are provided in the ATP III Guidelines At-A-Glance Quick Desk Reference, Step 9.

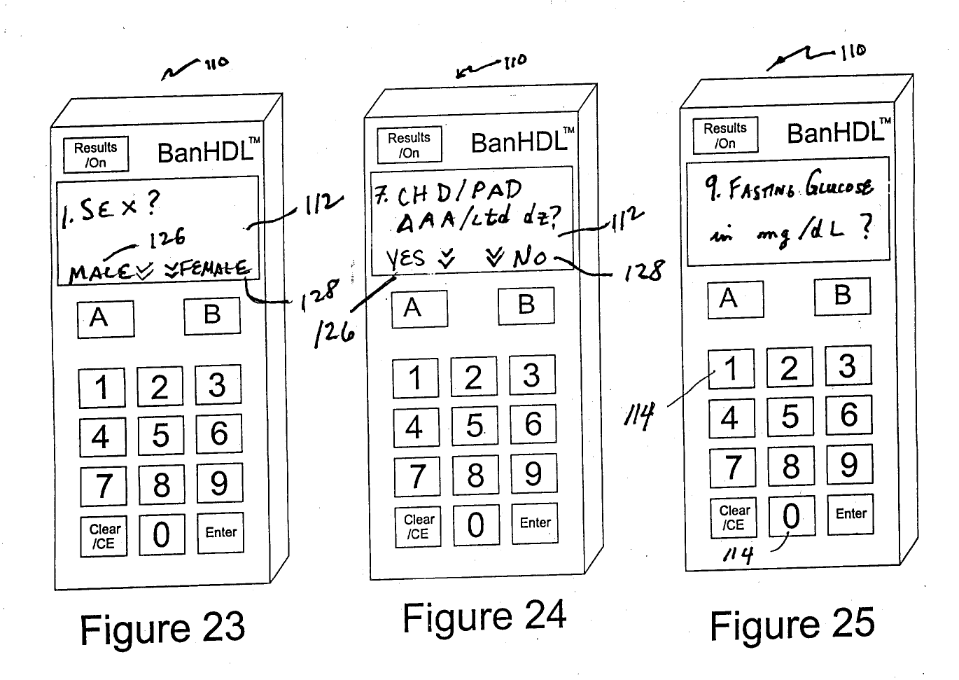

As noted above, the input device illustrated FIG. 3 may be used to practice the inventive method. In this case, special purpose keys 116 and 118 would be used to serve multiple functions and those functions would be labeled in areas 126 and 128 of screen display 112. For example, as illustrated FIG. 23, question number 1, “1. Sex?”, may be answered using special purpose keys 116 and 118 to indicate that the patient is male or female, respectively. As illustrated FIG. 23, areas 126 and 128, respectively, include the designations “Male” and “Female”, and these designations serve as labels for special purpose keys 116 and 118, respectively. Alternatively, the letters “M” and “F” may be used.

In similar fashion, as illustrated in FIG. 24, special purpose keys 116 and 118 may be labeled to perform as “Yes” and “No” keys. Here question number 7 appears on display 112 and the question may simply be answered by the physician with a yes or no has indicated in areas 126 or 128. It is noted that a pair of downward pointing arrows are used in regions 126 and 128 to indicate the position of the applicable special purpose keys 116 and 118.

In the case of questions requiring a numerical entry, such as question number 9, the special purpose keys 116 and 118 perform no function and the numerical data may simply be entered using the numerical keys 114. This is illustrated FIG. 25.

While an illustrative embodiment of the invention has been described, it is understood that various modifications will be apparent to those of ordinary skill in the art. Such modifications are within the spirit and scope of the invention which is limited and defined only by the appended claims.

1. A method of determining a regimen for the treatment of individuals with elevated risks of developing coronary heart disease, comprising receiving and electronically storing risk factor information respecting the sex, age, blood chemistry and lifestyle of an individual, electronically executing an algorithm on said risk factor information, and displaying at least one result of said execution of said algorithm on said risk information.

2. A method of reducing the risk of coronary heart disease in the general population, comprising receiving on a handheld computing device and electronically storing risk factor information respecting the sex, age, blood chemistry and lifestyle of an individual; electronically executing, on a dedicated electronic device, an algorithm on said risk factor information; and displaying at least one result of said execution of said algorithm on said risk information.

3. A method of reducing the risk of coronary heart disease in the general population, comprising presenting on a handheld computing device a series of questions relating to risk factors for coronary heart disease respecting the sex, age, blood chemistry and lifestyle of an individual,receiving on said handheld computing device and electronically storing risk factor information input by a physician; electronically executing, on a dedicated electronic device, an algorithm on said risk factor information; and displaying at least one result of said execution of said algorithm on said risk information in the form of a message identifying a potential therapy for consideration.

4. A method of reducing the risk of coronary heart disease in the general population, as in claim previous, further comprising funding distribution of dedicated electronic devices by selling advertising physically associated with said dedicated electronic devices.

5. A method of reducing the risk of coronary heart disease in the general population, as in claim previous, wherein said dedicated electronic devices are used by the physician while he is in the presence of his patient.

6. A method of detecting the likelihood of cardiac disease and treating the same, comprising:
(a) displaying on the screen of a hand-held device requests for data respecting lifestyle, family history, health, and blood parameters;
(b) inputting into random access memory, contained within said hand-held device, said data respecting lifestyle, family history, health, and blood parameters;
(c) performing an algorithm on said data in random access memory in response to requests for information; and
d) displaying on said screen results generated by the performance of said algorithm.
